(19) United States
(12) Patent Application Publication
(10) Pub. No.: US 2022/0400985 A1
(43) Pub. Date: Dec. 22, 2022

(54) System And Method For Tracking And Presenting Glucose Monitor Data

(71) Applicant: LifeScan IP Holdings, LLC, Beverly Hills, CA (US)

(72) Inventors: Allison Gonzales, San Jose, CA (US); Lisa Troncelliti, Ardmore, PA (US); Mark Hofmeister, Collegeville, PA (US); Bovornrat Cochard, Oakland, CA (US)

(73) Assignee: LifeScan IP Holdings, LLC, Beverly Hills, CA (US)

(21) Appl. No.: 17/351,643

(22) Filed: Jun. 18, 2021

(51) Int. Cl. A61B 5/145; A61B 5/00

(52) U.S. Cl. CPC A61B 5/14532; A61B 5/7475; A61B 5/743

(57) ABSTRACT

Systems and methods are provided for improved continuous glucose monitoring (“CGM”). The disclosed systems and methods provide for tracking analyte measurements, CGM-related event data, and sensor-related data. This data is combined and presented in connection with a graphical user interface that allows for improved functionality. Aspects of the disclosure allow for analyte measurements to be displayed in a graphical user interface that includes historical graphs of analyte measurements and incorporate event data as a plurality of event indications. The time frame of the historical graph can be altered by the user and the events can be presented a group within an event indication based on the time frame of the historical graph. The graphical user interface may also be configured to display graph trend data for each individual event as well as a sensor-life indication representing the period of time remaining before expiration of an analyte sensor.

### BACKGROUND OF THE INVENTION

Diabetes mellitus is a chronic metabolic disorder caused by an inability of the pancreas to produce sufficient amounts of the hormone insulin, resulting in the decreased ability of the body to metabolize glucose. This failure leads to hyperglycemia, i.e., the presence of an excessive amount of glucose in the blood plasma. Persistent hyperglycemia and/or hypoinsulinemia has been associated with a variety of serious symptoms and life threatening long term complications such as dehydration, ketoacidosis, diabetic coma, cardiovascular diseases, chronic renal failure, retinal damage and nerve damages with the risk of amputation of extremities.

Blood or interstitial glucose monitoring is required to achieve acceptable glycemic control. Continuous glucose monitoring (CGM) has been utilized over the last twenty years for such glucose monitoring. CGM creates data that is much more abundant and complex than that of traditional, episodic glucose monitoring. This added complexity may overwhelm users of the devices, as well as caregivers and health care practitioners (“HCPs”), especially in the absence of a suitable tool to assist in the interpretation of such data.

### BRIEF SUMMARY

The current disclosure provides for systems and methods relating to continuously monitoring analyte concentrations in a physiological fluid and for providing configured graphical user interfaces for tracking information relating to the analyte concentrations.

In accordance with aspects of the disclosure, systems and methods are provided for displaying analyte measurements on one or more devices that include a display, one or more memories for storing analyte measurement data and event data, and one or more processors. The one or more processors may be configured to: access analyte measurement data and event data from the one or more memories; determine a first time frame for which to display analyte measurement data as part of a graph; identify a plurality of events within the event data that occurred within the first time frame; determine, based on times associated with the plurality of events, whether a set of events are within a predetermined proximity in the graph having the first time frame; and provide for display the graph containing one or more event indications corresponding to the plurality of events, wherein a set of events are combined to be represented by a first event indication if it is determined that the set of events are within the predetermined proximity, and wherein each of the one or more event indications are associated with an analyte measurement within the graph.

In accordance with aspects of the disclosure, the one or more processors may be further configured to identify a first user input as corresponding to a command for the graph to transition from the first time frame to a second time frame, wherein the second time frame is a shorter period of time than the first time frame; determine that the set of events within the graph in the second time frame are outside of the predetermined proximity; and in response to the first user input, automatically provide for display the graph in the second time frame, wherein the set of events are displayed in connection with the graph as at least two separate event indications. The one or more processors wherein the one or more processors may be further configured to: identify a second user input as corresponding to a command for the graph to transition from the first time frame to a third time frame, wherein the third time frame is a longer period of time than the first time frame; determine that one or more additional events within the graph in the third time frame are within of the predetermined proximity of the set of events; and in response to the second user input, automatically provide for display the graph in the third time frame, wherein the set of events and the additional events are displayed in connection with the graph as a single combined event indication.

In yet other aspects of the disclosure, the single combined event indication may be associated with a single analyte measurement, and the events within the set of events may be associated with different times. The first event indication may be placed within the graph at a position corresponding to a median time period between the different time periods. The first event indication may include a symbol indicating the number of events with which it is associated.

In still other aspects of the disclosure, the one or more processors may be configured to identify user input selecting a particular event indication displayed with the graph; and in response to the selection the particular event indication, providing for display additional information relating to the one or more events associated with the event indication, wherein the additional information includes a trend graph of analyte measurements for each of the one or more events. Each trend graph may display a range of analyte measurements corresponding to predetermined periods of time before and after each of the one or more events.

In other aspects of the disclosure, the one or more processors may be further configured to: identify a sensor that is configured to provide the analyte measurements; determine a period of time for which the sensor will remain operational; and provide for display a sensor-life indication in the same display as the graph, wherein the sensor-life indication includes a number indicating the period of time for which a sensor will remain operational, and wherein the sensor life indication is color-coded to indicate the units of time represented by the number. The number may be surrounded by a plurality of ticker marks corresponding to the units of the time period represented by the number.

In yet other aspects of the disclosure, the one or more processors may be configured to compare a most-recent analyte measurement to one or more threshold values; and provide for display, within the graph, a current measurement indication having a color, wherein the color is based on the comparison of the analyte measurement with the one or more threshold values.

In still other aspects of the disclosure the one or more processors may be further configured to determine if the one or more processors are in communication with an operational sensor, and wherein the current measurement indication includes an animation if the one or more processors are in communication with an operational sensor. The one or more processors may be further configured to: identify a user selection of a particular point within the graph provided for display; determine an analyte measurement associated with the particular point; and provide for display a color-coded analyte measurement value, wherein the color corresponds to the relation of the analyte measurement value to one or more thresholds.

Other aspects of the disclosure provide for methods and systems for tracking and presenting sensor life data. For example, one or more processors may be configured to associate a first mobile device with a first user and a second mobile device with a second user; receive analyte measurements of the first user from an analyte sensor; receive analyte sensor data, wherein the analyte sensor data includes analyte measurements and sensor-life data indicating a period of time for which the analyte sensor will remain operational; receive, at one or more processors remote from the first mobile device and second mobile device, input data from the first user identifying the second user as having access to a selective subset of the analyte sensor data, wherein the selective subset of analyte sensor data includes sensor-life data; provide, by the one or more processors, the selective subset of analyte sensor data to the second mobile device; provide for display at the second mobile device the selective subset of analyte sensor data, wherein the display at the second mobile device includes a sensor-life indication that is based on the sensor-life data, the sensor-life indication including a sensor-life number indicating the period of time for which a sensor will remain operational, and wherein the sensor life indication is color-coded to indicate the units of time represented by the sensor-life number.

In accordance with other aspects of the disclosure, one or more processors may also switch the display of the sensor life indication at the second mobile device between a first mode and a second mode based on received input at the second mobile device. The sensor life indication in the first mode may consist of the sensor-life number and a plurality of ticker marks surrounding the sensor-life number. The plurality of ticker marks may include a set of solid ticker marks and a set of faded ticker marks, and wherein the number of solid ticker marks corresponds to the sensor-life number. When the analyte sensor will remain operational for less than an hour, one or more processors may provide for display a sensor-life indicator wherein the sensor-life number represents minutes and the plurality of ticker marks are replaced with a circular indicator that represents the amount of time that has elapsed in the final hour for which the sensor will remain operational. The second mode may comprise the sensor-life number as well as text identifying the date and time at which the analyte sensor will expire. The sensor life indication may include colors representing units of time corresponding to days, hours, and minutes. The one or more processors may include one or more servers in communication with the first mobile device and the second mobile device.

In accordance with other aspects of the disclosure, one or more processors may pair the first mobile device with a new analyte sensor; in response to the pairing, automatically transmitting from the first mobile device to the one or more processors updated sensor-life data; transmitting from the one or more processors to the second mobile device a notification that the first mobile device has been paired to a new analyte sensor; providing for display at the second mobile device an updated sensor-life indication based on the updated sensor-life data. The sensor-life indication may be presented as an overlay that remains visible as the second user transitions between a plurality of different displays on the second mobile device.

### BRIEF DESCRIPTION OF THE DRAWINGS

FIGS. 1 and 2 are diagrams of a continuous analyte monitoring system in accordance with aspects of the disclosure.

FIGS. 3A-5B are graphical user interfaces for visualizing data relating to analyte sensor measurements in accordance with aspects of the disclosure.

FIGS. 6-8 are representations of sensor-life indications in accordance with aspects of the disclosure.

FIGS. 9-16 are graphical user interfaces for visualizing data relating to analyte sensor measurements in accordance with aspects of the disclosure.

FIGS. 17 and 18 are flow diagrams depicting functions in accordance with aspects of the disclosure.

### DETAILED DESCRIPTION

The present disclosure relates to a continuous glucose monitoring system (a “CGM” system) in which an enhanced graphical user interface is provided. In a CGM system, glucose levels or concentrations can be determined by the use of a continuous glucose monitoring (CGM) sensor. The CGM sensor utilizes, for example, amperometric electrochemical sensor technology to measure glucose with electrodes such as a working electrode and a counter electrode, operably connected to the sensor electronics that are covered by a sensing membrane and a biointerface membrane, which are attached by a clip. Examples of such systems are found, for example, in U.S. Pat. No. 10,188,796 B2 and U.S. Patent Application Publication No. 2018/0296757 A1, each of which are herein incorporated by reference in their entirety.

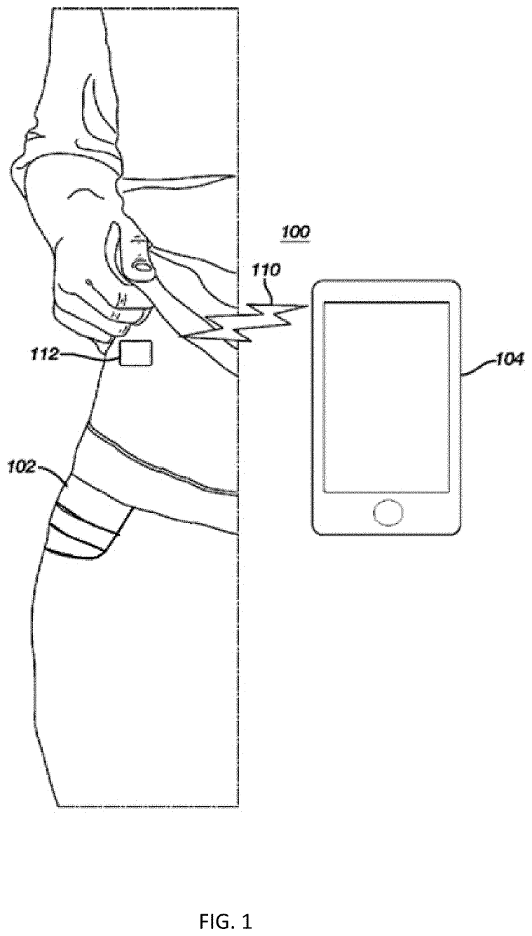

FIG. 1 depicts a system 100 for continuous glucose monitoring of a user 102. The system 100 includes an analyte (e.g., glucose) sensor 112, which transmits analyte concentration level values via wireless transmission 110 to a device 104. Device 104 may be a mobile device, such as a smart phone or a tablet, or any other wirelessly enabled mobile or fixed device that includes a display and at least one processor for receiving and processing data from the analyte sensor 112. Device 104, including the at least one processor thereof, may receive, store, and analyze data from the analyte sensor 112. In accordance with aspects of the disclosure, the graphical user interfaces of the system are configured to provide analyte concentration level values and related information in an improved manner over currently available systems. For example, the device may be smart phone, e.g., an iPhone available from Apple Inc., of California, and include an ARM microprocessor. In such a case the smart phone may be running a set of instructions that have been downloaded onto device 104 as an application or app so as to perform the functionality of device 104 described herein.

The analyte sensor 112 may be coupled with an electronics module that includes a wireless transceiver for facilitating communication with device 104. In another example, the sensor and transceiver may be part of a combined component. In one embodiment, the device 104 may include a touchscreen for input, and may run an operating system for hosting the graphical user interfaces described below. The analyte sensor 112 may be a continuous glucose monitoring sensor of any kind, such as those applied subcutaneously, transdermally, transcutaneously, including but not limited to implantable or other types. The continuous glucose monitoring sensor sends a data stream that includes the level of glucose concentration in the host 102. The device 104 is capable of receiving, storing, and processing this data stream. For instance, various algorithms known in the art may run on the at least one processor of device 104 to process the data stream, etc.

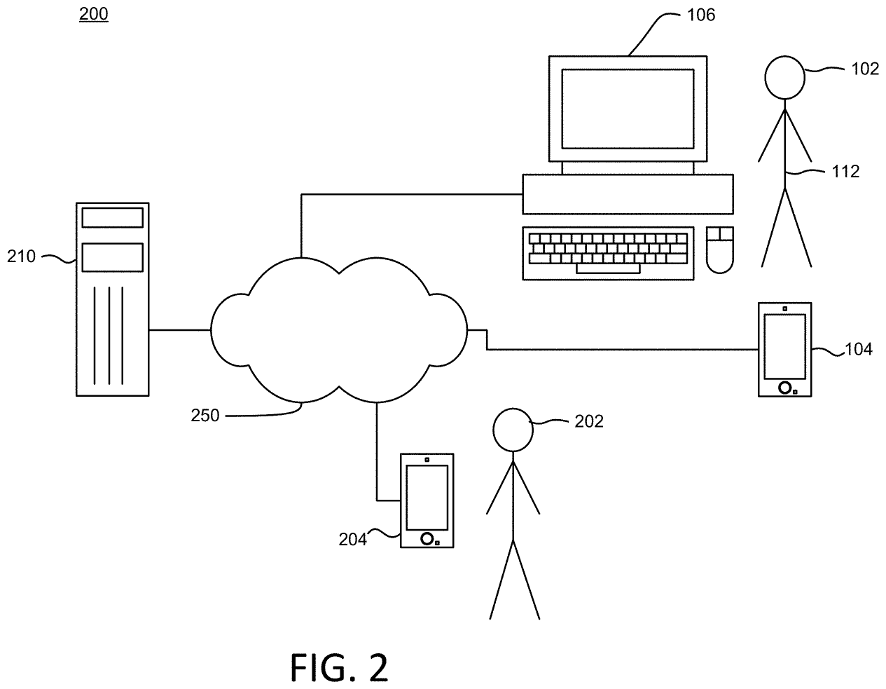

It should be understood that the specific glucose analyte measurement examples set forth herein are meant to illustrate a specific implementation and not limit the disclosure in any way. The techniques described herein may be used to visualize continuous analyte measurements for other configurations than that depicted in FIGS. 1 and 2, e.g., using other sensors for glucose or other analytes found in an interstitial fluid.

FIG. 2 is a diagram of system 200, which may include the analyte sensor 112 and device 104 of system 100. In addition, system 200 may include computer 210. The memory of computer 210 may store information accessible by one or more processors, including instructions and data that may be executed or otherwise used by the one or more processors. The memory of computer 210 or devices 104, 106, and 204 may be of any type capable of storing information accessible by the processor, including a computer-readable medium, or other medium that stores data that may be read with the aid of an electronic device, such as a hard-drive, memory card, ROM, RAM, DVD or other optical disks, as well as other write-capable and read-only memories. Systems and methods may include different combinations of the foregoing, whereby different portions of the instructions and data are stored on different types of media.

One or more processors of computer 210, as well as of devices 104, 106, and 204, may run sets of instructions. Instructions may be any set of instructions to be executed directly (such as machine code) or indirectly (such as scripts) by the processor. For example, the instructions may be stored as computer code on the computer-readable medium. In that regard, the terms “instructions” and “programs” may be used interchangeably herein. The instructions may be stored in object code format for direct processing by the processor, or in any other computer language including scripts or collections of independent source code modules that are interpreted on demand or compiled in advance. Functions, methods and routines of the instructions are explained in more detail below.

The data may be retrieved, stored or modified by the one or more processor in accordance with the instructions. For instance, although the system and method is not limited by any particular data structure, the data may be stored in computer registers, in a relational database as a table having a plurality of different fields and records, XML documents or flat files. The data may also be formatted in any computer-readable format.

The processors of computer 210 and devices 104, 106, and 204 may be any conventional processor, such as processors from Intel Corporation or Advanced Micro Devices. Alternatively, the processor may be a dedicated device such as an ASIC. Memory may be a hard drive or other storage media located in a server farm of a data center. Accordingly, references to a processor, computer, or memory will be understood to include references to a collection of processors, computers, or memories that may or may not operate in parallel.

The computer 210 may be at one node of a network 250 and capable of directly and indirectly communicating with other nodes of the network. For example, computer 210 may include a web server that is configured to communicate with devices 104, 106, and 204 via network 250 so as to transmit and display information to a user, such as users 102 or 202 of FIG. 2, on displays of devices 104, 106, and 204. Computer 210 may also comprise a plurality of computers that exchange information with different nodes of a network for the purpose of receiving, processing and transmitting data to client devices. In this instance, the client devices will typically still be at different nodes of the network than any of the computers comprising server 210.

Network 250, and intervening nodes between server 210 and client devices, may comprise various configurations and use various protocols including the Internet, World Wide Web, intranets, virtual private networks, local Ethernet networks, private networks using communication protocols proprietary to one or more companies, cellular and wireless networks (e.g., WiFi), instant messaging, HTTP and SMTP, and various combinations of the foregoing. Although only a few computers are depicted in FIGS. 1-2, it should be appreciated that a typical system can include a large number of connected computers.

Devices 104, 106, and 204 may be configured similarly to the server 210, with one or more processors, and memory containing stored instructions. Devices 104, 106, and 204 may have all of the components normally used in connection with a computer such as a central processing unit (CPU), memory (e.g., RAM and internal hard drives) storing data and instructions such as a web browser, an electronic display, and user input. The client device may also include a camera, GPS receiver, speakers, a network interface device, as well as all of the components used for connecting these elements to one another. In addition, each device 104, 106, 204 may be a device intended for use by one or more particular users. For example, devices 104 and 106 may be associated with user 102, while device 204 is associated with user 202. The association of a user to a device may be achieved by assigning each user with a unique identifier, such as a user ID, and by requiring the user to verify his or her identity by providing verification prior to accessing an application or certain information with one or more applications that are running on the device. User verification may, for example, take the form of a password or a unique biometric identification.

In accordance with aspects of the disclosure, user 102 may be provided with CGM data via device 104 that includes an interactive trend graph, event indications, event logs, as well as sensor operation data. As set forth further below, the interactive trend graph, event indications, and sensor operation data provide an enhanced user interface allowing for flexibility and simplicity in acquiring comprehensive CGM data.

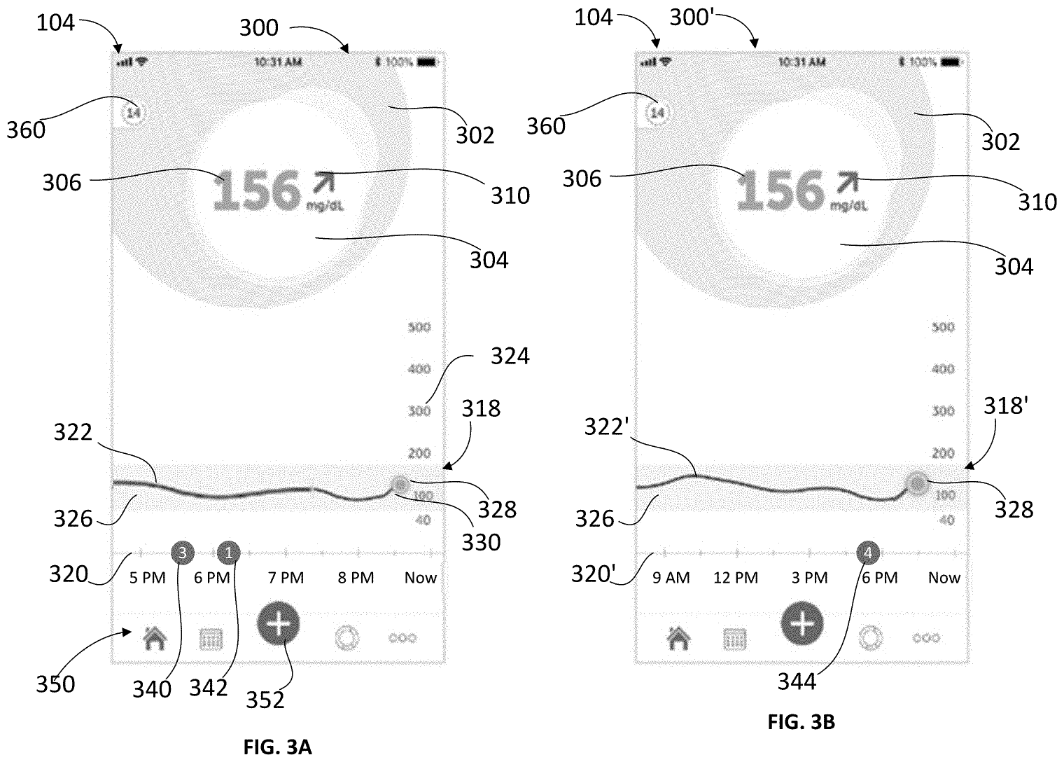

FIGS. 3A and 3B are graphical user interfaces 300 and 300′, respectively, as presented on the display of device 104 of FIGS. 1 and 2. In an upper portion, the graphical user interface 300 includes a colored bubble 302, a white bubble 304, a current analyte measurement value 306, a trend indicating arrow 310, and a sensor-life indication 360. The color of the colored bubble 302 may be configured to change in order to represent whether the current analyte concentration level is above, below, or at the target range of analyte concentration levels, as determined by one or more processors of device 104. In some embodiments, a determination that the level is at a target range would be based on the measured level being within an upper threshold value and a lower threshold value. Levels below the target or target range, as here, are represented by a blue colored bubble, levels above the target analyte concentration level are represented by a red colored bubble, and levels at the target analyte concentration level are represented by a green colored bubble. The white bubble 304, as shown in the herein depicted embodiment, is static and includes near the center of the bubble 304, a current analyte concentration level numerical value. In other embodiments, the concentration level may be depicted using other visual cues, such as by a cluster of dots or objects, a bar of a bar chart embedded in the bubble, a number located in a different portion of the bubble, and the like. As discussed further below, sensor-life indication 360 is a graphic overlay that provides information regarding the period of time that the analyte sensor will remain operable. The graphical user interface 300 also includes an arrow indicator 310, which may include a single arrow, double arrow, blinking arrow(s), and any other directional indicator. In some examples, the arrows may also move to indicate the directional trend of the most recent analyte measurements.

The lower portion of interface 300 includes an historical graph 318 of analyte measurements. This historical graph may include a timeline 320, graphical analyte measurements 322, analyte value axis labels 324, a target analyte concentration band 326, and a current analyte reading animation 328. The analyte measurements 322 of the historical graph 318 correspond to particular measurements that have been made by the analyte sensor described above, and these graphical analyte measurements 322 are displayed within historical graph 318 in a manner that indicates the corresponding time at which each analyte measurement was taken, as provided by timeline 320.

The current analyte reading animation 328, which may also be referred to as a “now dot”, may be configured to pulsate between the depiction of FIG. 3A and FIG. 3B. In one or more embodiments, current analyte reading animation 328 (or “now dot”) can stop pulsating or disappear to indicate an error or lack of connectivity with the analyte sensor. In one or more embodiments, the now dot may include a halo 330 that surrounds the central dot, and only the halo 330 may pulsate. The now dot 328 can also change colors, flash at different rates, etc., to where the colors are rates are indicative of conditions such as analyte concentration levels with respect to predetermined threshold concentrations. For example, in FIG. 3A, the now dot 328 is green, as the current analyte measurement value is within upper and lower threshold values that define analyte concentration band 326. Now dot 328 can change color if the current analyte value is outside of the analyte concentration band 326, for example, now dot 328 may be displayed in red if it is above analyte concentration band 326 and may be displayed in blue if it is below analyte concentration band 326. In addition, an icon can be further presented in the graphical user interface 300 to indicate the loss of connectivity with an analyte sensor. In other embodiments, a broken link icon may be displayed after some period of absent data, e.g., one minute. In such a case, a broken link icon may indicate loss of connection or indicate that the sensor is connected but is delivering unstable (e.g., invalid or nonactionable) readings.

Timeline 320 of interface 300 also includes event indications 340 and 342. Event indications 340 and 342 represent CGM-relevant events that have occurred, such as information relating to physical activities (e.g., sports, walking, running, swimming, etc.), dietary information (e.g., identification of meals, type of foods consumed, amount of carbs consumed, etc.), insulin information (e.g., amount and timing of insulin taken), as well as general user notes regarding other events that could affect CGM readings. The user can select icon 352 from the navigation bar 350 at the bottom of interface 300 to enter events into device 104. Once an event has been input into device 104, the timeline 320 may be updated to include an indication of the event. Each event indication displayed on timeline 320 may be associated with a time and an analyte measurement.

If multiple events are associated with a particular time, the event indication that is displayed on the timeline may be configured to indicate this fact. For example, in FIG. 3A event indication 340 contains the number “3”, while event indication 342 contains the number “1”. The “3” within event indication 340 represents the number of events that are associated with that event indication. Each event indication 340 and 342 may be configured to be a selectable icon. As discussed further below, upon selection of an event indication by the user, device 104 may display an event log that provides detailed information about the one or more events that are associated with the selected event indication.

The time frame for historical graph 318 may be set to a particular default value. Timeline 320 of FIG. 3A indicates that historical graph 318 has a time frame that spans 4 hours. However, the default time frame for device 104 could be set to one of a plurality of different time frames, such as 1 hour, 4 hours, 8 hours, 12 hours, 24 hours, or longer. The ability to edit the default time frame may be presented in a home screen in which the user may enter the desired default time frame for historical graph 318.

Device 104 may also be configured to adjustably vary the time frame of timeline 320 in accordance with one or more user inputs. For example, device 104 may have a touch-sensitive display that is capable to recognizing a user input in which the user pinches two fingers together while touching a portion of the display screen that is at or near historical graph 318. Upon receiving this user input, device 104 may modify historical graph 318 to correspond to a new time frame in accordance with the pinch command For example, a user of device 104 may provide a pinch command to the interface 300 shown in FIG. 3A. In response to this pinch command, device 104 can alter the displayed interface 300 to that of graphical user interface 300′ shown in FIG. 3B. Graphical user interface 300′ has been altered to include a modified historical graph 318′ that includes a modified set of graphical analyte measurements 322′ corresponding to a modified time frame for timeline 320′. In particular the time frame for timeline 320′ is now approximately 12 hours, rather than the 4 hour time frame for timeline 320 of interface 300. The amount of change in the time frame can be based on the size or number of pinch commands that the user provides. In addition, device 104 may have settings whereby the pinch command causes a smooth transition from one time frame to another or the pinch command causes historical graph 318 to jump from one time frame to another. For example, device 104 could be set so that pinch commands cause a timeline with a 4 hour-time frame to automatically jump out to 8-hour, 12-hour, or 24-hour time frames. A user input that causes device 104 to transition from a shorter time frame to a longer time frame can be referred to as “zooming out” of a timeline, while a transition from a longer time frame to a shorter time frame can be referred to as “zooming in” on a timeline.

In displaying the historical graph of analyte measurements, device 104 may be configured so as to present event data in accordance with the context of the time frame of the historical graph. For example the event indications 340 and 342 of FIG. 3A have been combined within timeline 320′ of FIG. 3B to a single event indication 344. Event indication 344 now displays the number “4”, indicating that it corresponds to four distinct events. In determining whether events are to be combined into a single event indication, device 104 may identify whether a set of events are within some predetermined proximity to one another within the time frame of the timeline that is being displayed. When one or more events are determined to be within the predetermined proximity for a given time frame, the events may be displayed as part of a single event indication.

The predetermined proximity may be based on the distance between event indications, if a set of events were displayed using two or more separate event indications on the timeline. Each event may be associated with a time or a time range, which determines the position of the event within the timeline. If multiple events are recorded at the same time or at overlapping times, then device 104 may present these events on a timeline using a single event indication. For example, In FIG. 3A, event indication 340 represents three events all of which are associated with the same time. As can be seen in timeline 320, the time of the events associated with event indication 340 is a time between 5 PM and 6 PM. With regard to event indication 342, it is associated with an event that occurred shortly after 6 PM. With respect to timeline 320, event indications 340 and 342 have a sufficient distance between one another that the event associated with event indication 342 may be displayed separately from event indication 340. However, in FIG. 3B, timeline 320′ has a time frame that would require event indications 340 and 342 to be within a predetermined proximity to one another. Based on this determination, device 104 combines the events associated with event indications 340 and 342 into a single event indication 344.

Identifying events that are within a predetermined proximity may be based on a determination that if separate event indications were placed on the timeline at times corresponding to those events, then the event indications would touch or overlap one another. For example, the four events associated with event indication 344 are associated with times that would produce overlapping event indications, if each event were given its own indication. Based on this determination, device 104 displays the four events with a single event indication 344. Thus, in response to a user input for device 104 to zoom out of timeline 320 of FIG. 3A to timeline 320′ of FIG. 3B, event indications 340 and 342 may be combined into a single event indication 344. The predetermined proximity may also take the form of a particular range of time relative to the overall time frame of the timeline. This range of time may be correlated to the length of the time frame. For example, with respect to timeline 320 of FIG. 3A, the predetermined proximity between events may be a time range of 20 minutes, so that any events within 20 minutes of each other are displayed in connection with a single event indication. With respect to timeline 320′ of FIG. 3B, the predetermined proximity may be one hour, so that any events within 1 hour of each other are displayed in connection with a single event indication.

In addition, the user of device 104 may provide a user input corresponding to a “zoom in” command, such as by providing a reverse pinch touch command As device 104 zooms into a timeline, an event indication that is associated with multiple events may be separated into two or more event indications. For example, a user may provide a reverse pinch touch command to device 104, as it displays interface 300′ of FIG. 3B so that device 104 transitions to interface 300 of FIG. 3A. In making this transition, device 104 may determine that one or more events of event indication 344 will no longer be within the predetermined proximity to one another when displayed in timeline 320. Based on this determination, event indication 344 may be divided into two or more separate event indications 340 and 342. The historical graphs may also be scrollable in response to a user input. For example, a user may swipe right on the touch display of device 104 in order to scroll through historical graph 318 of FIG. 3A, so as to view previous analyte measurements, while remaining in the four-hour time frame. The historical graph may be configured to be scrollable within a predetermined period of time, such as the last 24 hours.

Each event that is stored or accessed by device 104 may be associated with an analyte measurement, as well as a time. The historical graph 318 of the graphical user interface is configured so that the user is able to identify the manner in which CGM-related events have affected the user's analyte measurements over time. Thus, event indications are displayed within historical graphs in a manner that allows the user to identify the relative timing of events with respect to the user's analyte measurements. When grouping events into a single event indication, the disclosed system may identify the range of times for the grouped events and identify a median location based on that range of times. This median location may be based on an average of the times for the grouped events, or may be based on a mid-point between two of the events. For example, device 104 may identify the earliest and latest events within a set of events that are going to be grouped together into a single event indication. Device 104 may then identify the mid-point in time between the earliest event and latest event as being the point on the timeline at which the event indication will be placed. Displaying a group of events in this manner will allow a user to be able to view the historical graph in longer time frames while still identifying the general trend of analyte measurements in connection with those events.

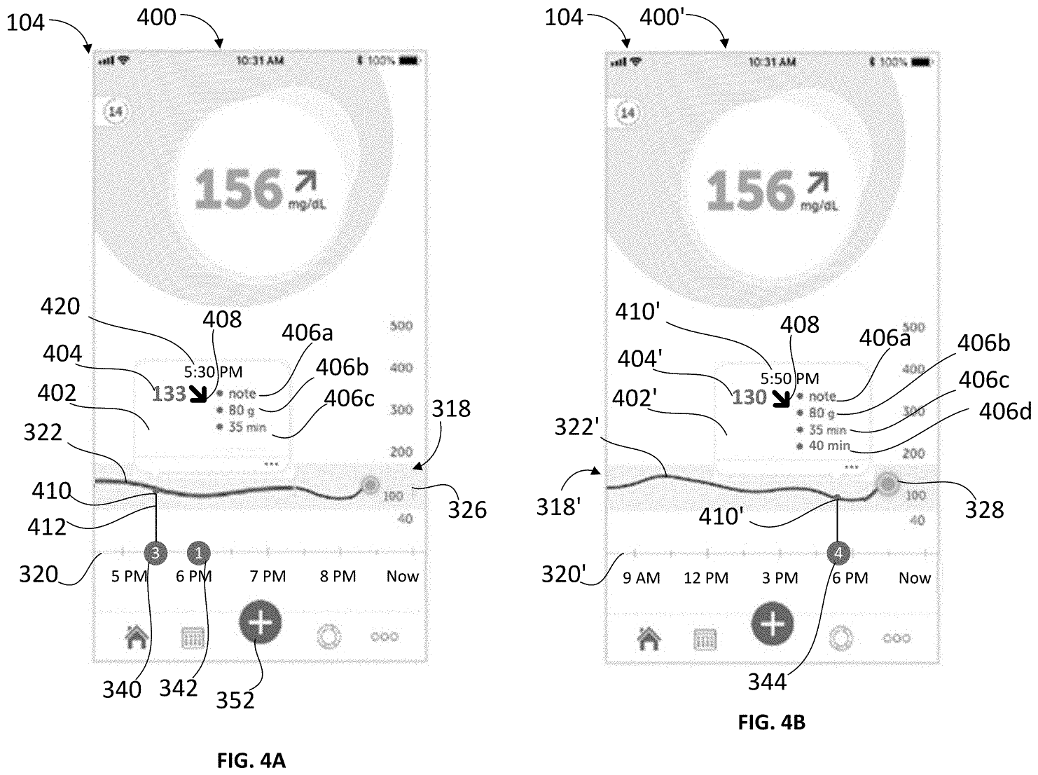

The user may also select portions of the historical graph 318 in order to view analyte measurements and event data in connection with specific times. The user selection may occur by touching or dragging the user's finger along a region of graphical analyte measurements 322. For example, FIGS. 4A and 4B include graphical user interfaces 400 and 400′ in which a user has selected a point within the historical graph 318. In FIG. 4A, the user has last touched a portion of historical graph 318 in which graphical analyte measurements 322 are associated with event indication 340. Point 410 indicates the graphical analyte measurement 322 that has been selected, and line 412 extends down to timeline 320 so as to indicate the time of the selected analyte measurement 322. In one or more embodiments, point 410 may be color-coded based on the current graphical analyte measurement that it is representing. For example, measurements within analyte concentration band 326 may be displayed with a green point 410, while measurements above and below analyte concentration band 326 may be displayed with red and blue points 410, respectively. In FIG. 4A, the point 410 and line 412 indicate that the selected analyte measurement is associated with event indication 340. A graphical overlay 402 may also be displayed in connection the selected point 410. Graphical overlay 402 may include the analyte measurement value 404, trend arrow 408, and time 420 that corresponds with the selected point 410. Analyte measurement value 404 may be color-coded to indicate whether the selected point corresponds to an analyte measurement that is above, below, or within a designated range of analyte values. For example, analyte measurement value 404 may be presented in green, as point 410 corresponds to an analyte measurement value 404 that is within target analyte concentration band 326. Values above target analyte concentration band 326 may be presented, for example, in red, while values below target analyte concentration band 326 may be presented in blue. Trend arrow 408 is presented to indicate the manner in which the analyte value is changing at point 410.

Device 104 may also determine that selected point 410 corresponds to analyte measurements that have been associated with event indication 340. Based on this determination, graphical overlay 402 may include event descriptions 406a-c which provide information identifying the nature of the one or more events for which event indication 340 is associated. For example, event description 406a represents that the user has entered a note, while event description 406b represents an event in which the user has consumed 80 grams of carbs, and event description 406c represents the occurrence of 35 minutes of a physical activity. The graphical user interface may be configured so as to allow the user to select what type of information is displayed within the graphical overlay 402. For example, the user may set the graphical overlay 402 to only display analyte measurement data, without displaying event descriptions 406a-c. As set forth below, event information can be accessed through the event log, even if it is not displayed in connection with graphical overlay 402.

In FIG. 4B, the user has selected point 410′ that corresponds to event indication 344. Accordingly, a similar graphical overlay 402′ is presented as the graphical overlay 402 discussed in connection with FIG. 4A. As can be seen in FIG. 4B, graphical overlay 402′ now contains four event descriptions 406a-d. In addition, given that the mid-point of the four events associated with event indication 344 is different than the mid-point of the three events associated with event indication 340, the time 410′ and analyte measurement value 404′ displayed in graphical overlay 402′ are slightly different than those displayed in graphical overlay 402 of FIG. 3A.

If the user wishes to view event indications that are each positioned at the exact time of individual events, the user may zoom into the timeline until all of the event indications have been divided to represent individual events, wherein the historical graph can be viewed to show more granular data regarding the effect of each event on the recorded analyte measurements.

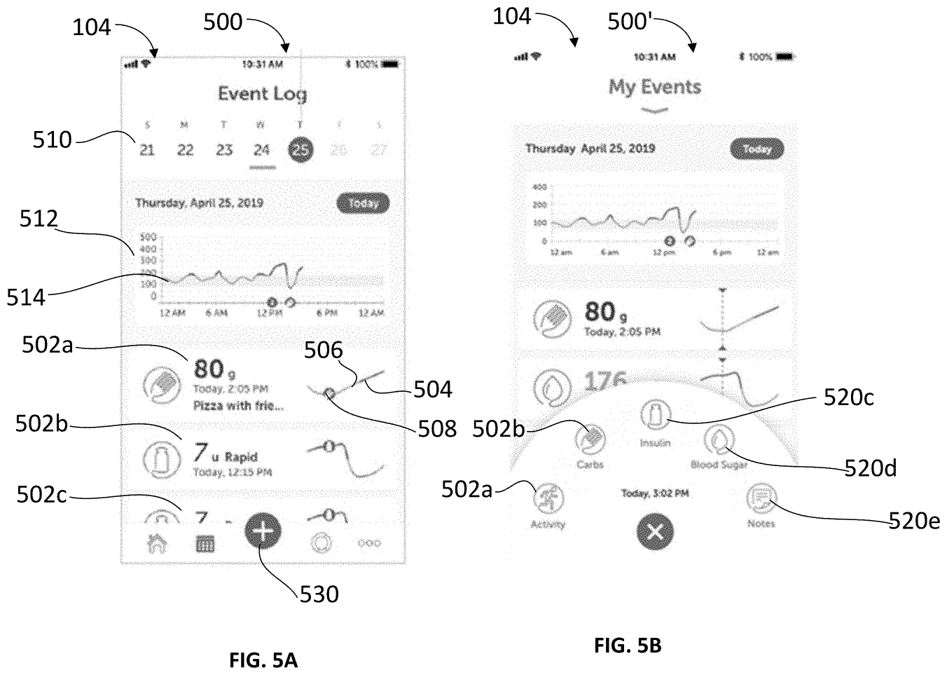

In addition to, or alternatively to, zooming into the historical graph, the user may receive more granular event data by selecting the graphical overlay 402 that appears in connection with selected point 410 so as to bring up a display of an event log. For example, FIG. 5A is graphical user interface 500 displaying an event log for a patient. The event log may include a calendar 510 with selectable dates, a daily historical graph 512 for displaying a graph of analyte measurements taken on a particular day, and a scrollable list of event listings 502a-c. Historical graph 512 may appear as with a color-coded line 514 so that analyte measurement values appear in different colors within the graph, depending on if the values are within a target range (e.g., appear in green), above the target range (e.g., appear in red), or below the target range (e.g., appear in blue). Each event listing 502a-c may provide information identifying the type of event, the time associated with the event, as well as other CGM-relevant data associated with the event. The event listings may be presented in reverse chronological order.

In addition, each event listing 502a-c may include its own trend graph 504. A trend graph 504 may include a graphical representation of analyte measurements 506 as well as an icon 508 representing the time at which the event occurred relative to the graphical representation of analyte measurements 506. Icon 508 may take the form of any graphical indication or marking that designates the occurrence of the event within the graphical representation of analyte measurements 506. The graphical representation of analyte measurements 506 may be color-coded so that portions of the graphical representation indicate wither the analyte measurements are above, below, or within a target range of analyte values. For example, in trend graph 504, the left-hand side of the graphical representation 506 may appear in blue, indicating that this portion is below the target range of analyte values; the center portion of the graphical representation 506 may appear in green, indicating that this portion is within the target range of analyte values; and the right-hand portion of the graphical representation may appear in red, indicating that this portion is above the target range of analyte values.

While analyte measurements may be presented in connection with the trend graph 504, the shape and/or color of the trend graph 504 is sufficient to indicate how the particular event has effected the analyte measurements. In particular, the trend graph 504 may be configured so that it contains a plurality of analyte measurements that occurred before the event, as well as plurality of analyte measurements that occurred after event. The amount of analyte measurements before and after the event may be based on a predetermined period of time or a predetermined number of measurements. The period of time prior to the event can be selected to be of a sufficient amount of time so as to indicate whether the user's analyte measurements were going up, going down, or were stable prior to the event. The period of time after the event can be selected to be of a sufficient amount of time so as to indicate whether the event had an effect on the user's short-term analyte measurements. For example, trend graph 504 contain a graphical representation of analyte measurements 506 that corresponds to the period of time between one hour prior to the event and two hours after the event. Thus, each event listing 502a-c may have its own unique trend graph 504 that corresponds to a period of time around that particular event. In this way, the user may access the event log in to determine the effect of particular events on a subset of the user's analyte measurements, while using the zoomed out view of the historical graph 318 to determine the general effect of multiple events on the user's analyte measurements over a longer period of time.

Interface 500 may also include icon 530, which can be selected by the user in order to add additional event data to the event log. For example, FIG. 5B is an interface 500′ that may be displayed by device 104 when icon 530 is selected. In interface 500′ a plurality of event icons 520a-e are displayed, with each event icon representing a different type of event. The user may select the appropriate event and then enter data relating to that event, including identification of the time, or range of times, at which the event occurred. Returning to FIGS. 3A-B, upon adding an event to the event log, the device 104 may update the historical graph 318, so as to include the added event within one of the event indications displayed on the timeline 320.

In addition to the features described above, device 104 of FIGS. 1 and 2 may also receive sensor-life data from analyte sensor 112. In particular, analyte sensors, such as sensor 112, will have a specific date and time at which the sensor will expire. Expiration of a sensor without replacement can result in gaps in the patient's analyte measurement data and can detrimentally impact the ability of the user and the user's physician to track the patient's condition. Thus it is important for the user 102 and for others to be able to track the sensor life of analyte sensor 112. In accordance with embodiments disclosed herein, the graphical user interface of devices 104 and 204 may provide user 102 and user 202 with a quick, persistent, and non-intrusive indication of the sensor-life data for analyte sensor 112.

For example, graphical user interface 300 of FIG. 3A contains an overlay of a sensor-life indication 360 that indicates the time period that is remaining for the user's analyte sensor. Specifically, the number displayed within sensor-life indication 360 may indicate the number of days, hours, or minutes that are remaining before the analyte sensor will expire. The units of time (e.g., days, hours, or minutes) that the number represents may be represented by at least a portion of the sensor-life indication 360 being color-coded, for example, the number within sensor-life indication 360 may appear in black when the number represents days remaining in the sensor life, while the number may appear in red when the number represents hours or minutes remaining in the sensor life.

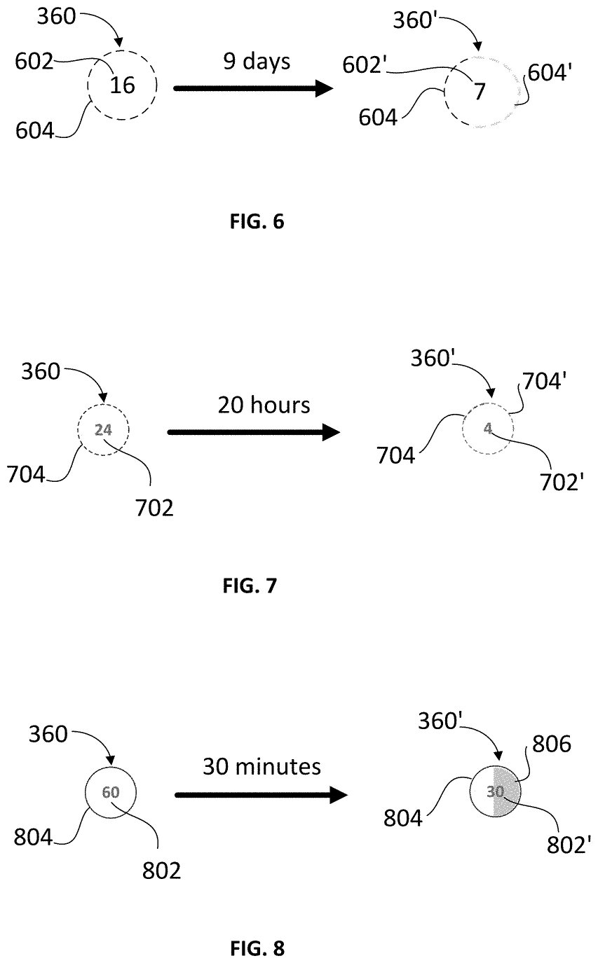

The period of time remaining before expiration of the sensor may also be indicated by graphical indications that surround the displayed number in sensor-life indication 360. For example, in FIG. 6, sensor-life indication 360 is providing sensor-life data in units of days. Sensor-life indication 360 contains ticker markers 604 that change in appearance in accordance with the number of days remaining before sensor expiration. On the left-hand side of FIG. 6, sensor-life indication 360 contains a displayed-number 602 as well as a plurality of ticker marks 604 that surround displayed number 602. Each day, the displayed-number 602 will count down the remaining days before expiration of the sensor. The displayed-number 602 is displayed in black, indicating that it represents days, rather than hours or minutes. In addition, the appearance of one ticker mark 604 will change in connection with the number of remaining days. For example, the right-hand side of FIG. 6 is sensor-life indication 360′, which represents the appearance of sensor-life indication 360 after nine days have passed. The displayed-number 602′ is now a “7”, representing that there are seven days of sensor life remaining, and nine of the ticker marks 604′ appear with a light gray color, while seven ticker marks 604 continue to be displayed in bold. Accordingly, sensor-life indication 360, 360′ provides the user with both a numeric and graphical representation of the days that remain before the sensor expires.

In FIG. 7, sensor-life indication 360 is providing sensor-life data in units of hours. On the left-hand side of FIG. 7, sensor-life indication 360 contains a displayed-number 702 as well as ticker marks 704. The displayed number 702 is a red “24” representing that there are 24 hours before the sensor expires. Each hour, the displayed-number 702 will count down the remaining hours before expiration of the sensor. In addition, sensor-life indication 360 contains 24 ticker marks 704, which appear in red and surround displayed-number 702. The appearance of one ticker mark 704 will change each hour. For example, the right-hand side of FIG. 7 is sensor-life indication 360′, which represents the appearance of sensor-life indication 360 after 20 hours have passed. The displayed-number 702′ is now a red “4”, representing four hours of sensor life remaining. In addition, a plurality of ticker marks 704′ appear in gray, and only four ticker marks 704 remain in red. Thus, sensor-life indication 360, 360′ of FIG. 7 provides the user with both a numeric and graphical representation of the hours that remain before the sensor expires.

In FIG. 8, sensor-life indication 360 is providing sensor-life data in units of minutes. On the left-hand side of FIG. 8, sensor-life indication 360 contains a displayed-number 802 that is surrounded by a solid circle 804. The displayed number 802 is a red “60”, which in combination with the solid circle 804, represents that there are 60 minutes before the sensor will expire. Each minute, the displayed-number 802 will count down the remaining minutes before expiration of the sensor. In addition, sensor-life indication 360 may include a pie graph indicating the proportion of the last hour of sensor life that has passed. For example, the right-hand side of FIG. 8 is sensor-life indication 360′, which represents the appearance of sensor-life indication 360 after 30 minutes have passed. The displayed-number 802′ is now a red “30”, representing 30 minutes of sensor life remaining. In addition, sensor-life indication 360′ contains pie graph 806. Pie graph 806 is half filled, representing that half of the last hour of sensor life has passed. Thus, sensor-life indication 360, 360′ of FIG. 8 provides the user with both a numeric and graphical representation of the minutes that remain before the sensor expires.

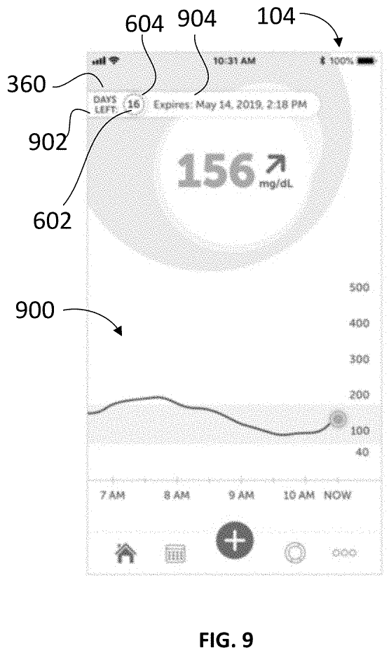

In accordance with embodiments of the disclosure, the sensor-life indication 360, 360′ of FIGS. 6-8 may be displayed as an overlay at a particular portion of the display screen of device 104, such as is shown in FIGS. 3A-B. By displaying a numeric and graphical representation of the remaining sensor-life time period in the manner described above, the sensor-life indication 360 may be presented as a small overlay that is non-obtrusive and that does not obscure other relevant information that is being provided on the graphical user interface. For example, the use of color-coding, tick markers, and pie charts to designate units of time allows for the sensor-life indication 360 to provide the user with the remaining time period without requiring the display of text beyond a single number. However, in one or more embodiments, sensor-life indication 360 may be configured to switch from compact mode, as shown in FIG. 3A, to an expanded mode, as shown in FIG. 9.

Device 104 may receive a user input to toggle sensor-life indication 360 between the compact mode and the expanded mode. For example, the user may tap on sensor-life indication 360 of FIG. 3A, and in response to this user input, device 104 may present sensor-life indication 360 in an expanded mode, as shown in graphical user interface 900 of FIG. 9. In the expanded mode, sensor-life indication 360 may continue to display ticker marks 604 and displayed-number 602, but may also include a unit indication 902, as well as an expiration time 904. The unit indication 902 may include text that identifies the units that are being represented by displayed-number 602. For example, unit indication 902 may include the text “DAYS LEFT”, “HOURS LEFT”, or “MINUTES LEFT” depending on the unit to time that is being represented by displayed-number 602. Expiration time 904 may include identification of the month, day, year, and time of sensor expiration, as is shown in FIG. 9. The user of device 104 may collapse expanded sensor-life indication 360 back to the compact mode by tapping the expanded sensor-life indication. In one or more embodiments, the expanded sensor-life indication may automatically collapse after a predetermined period of time, such as 5 seconds.

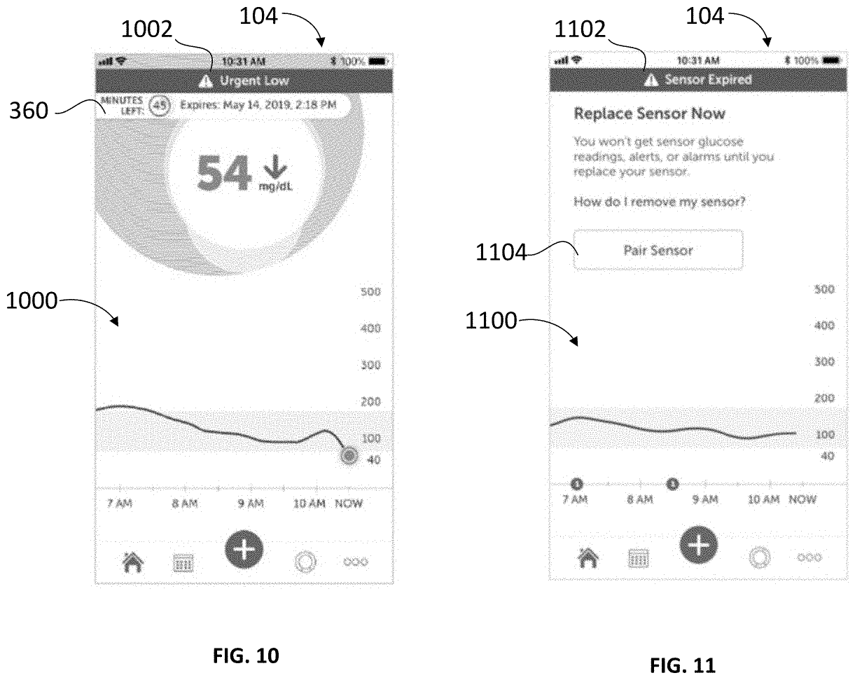

When the sensor-life is determined to be within some predetermined time frame, device 104 may also display a low sensor-life warning, such as the “Urgent Low” banner 1002 shown in graphical user interface 1000 of FIG. 10. When the analyte sensor has the expired, device 104 may display a home screen message stating that the sensor has expired, and the sensor-life indication 360 may no longer be displayed. For example, FIG. 11 is a graphical user interface 1100 that displays a message that the sensor has expired and needs to be replaced. This message may include a banner 1102. In addition, device 104 may display a selectable icon 1104 that may be used to replace the expired sensor with a new sensor. For example, a user may put on a new analyte sensor and tap icon 1104. In response to tapping icon 1104, device 104 may search for and pair with the new analyte sensor. Upon paring with a new analyte sensor, device 104 may receive sensor-life data for the new analyte sensor. The received sensor-life data may then be used to generate a sensor indication 360 that is based on the expiration date and time of the new analyte sensor.

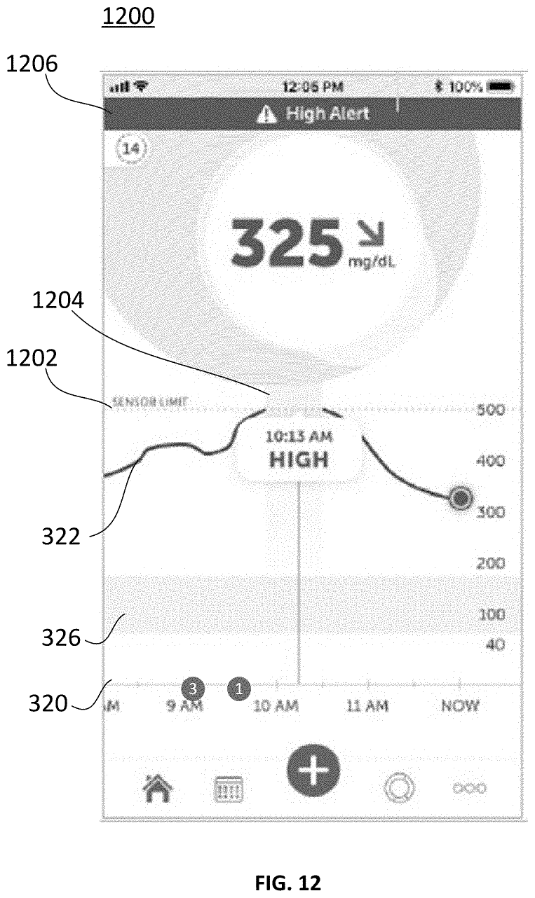

The disclosed system may also determine if the analyte sensor 112 has exceeded an upper or lower sensor limit. When an analyte sensor 112 is above or below the sensor limit, device 104 may provide a display indicating that the sensor is over the sensor limit and an accurate reading cannot be presented. For example, in FIG. 12, graphical analyte measurements 322 do not appear above sensor limit 1202. Instead, graphical user interface 1200 displays a vertical band 1204. The width of vertical band 1204 corresponds to the period of time over timeline 320 for which the analyte sensor was at or above the sensor limit. The vertical band 1204 may be presented with a vertical color gradient, as shown in FIG. 12. Vertical band 1204 may be color-coded, for example vertical band 1204 may be red when the analyte sensor is above an upper sensor limit, and vertical band 1204 may be blue when the analyte sensor is below a lower sensor limit.

Device 104 may also display color-coded push notifications in connection with high and low sensor readings. For example, push notifications may appear with a blue icon if analyte sensor measurements are below a lower target threshold, and push notifications may appear with a red icon if analyte sensor measurements are above an upper target threshold. These target thresholds may be set by the user using the graphical user interface of device 104.

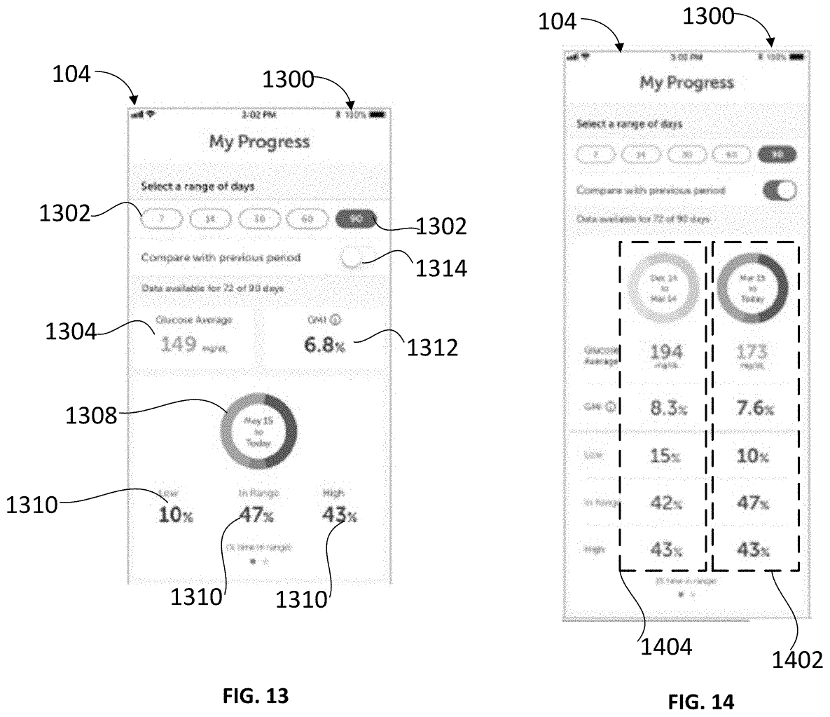

FIG. 13 provides a graphical user interface 1300 that may be referred to as a “My Progress” screen, and which may be displayed on device 104 so as to provide the user with a summary of his or her analyte measurements over a desired period of time. Graphical user interface 1300 may include a plurality of icons 1302 that represent different time frames, for example, 7, 14, 30, 60, and 90 days. The user may select one of the icons 1302 in order to view a summary of the user's analyte measurements over the selected period of time. In FIG. 13, the user has selected icon 1302 corresponding to a 90-day summary Accordingly, graphical user interface provides a glucose average 1304 corresponding to the user's average glucose level over the 90-day period. Also included in the display are percentage values 1310 representing the percentage of time over the 90-day period that the user has been within, below, or above the user's desired target range of analyte values. The user may also select an alternative setting, wherein the percentage values 1310 are presented as absolute values of time (e.g., hours and minutes).

A graphical representation 1308 of these percentages is also provided within the display. As described above, the same color-coded may be used for the graphical representation 1308 and percentage values 1310, with green representing values within the target range, red representing values above the target range, and blue representing values below the target range. In addition, the graphical user interface 1300 may also display the user's Glucose Management Indicator percentage over the desired range of time.

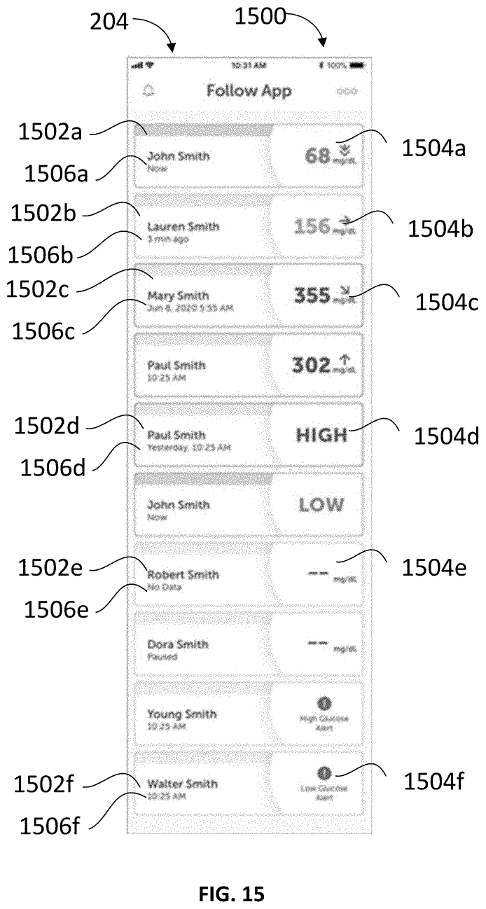

The information presented in graphical user interface 1300 for a particular period of time may also be presented along with a previous period time, so that the user may compare his or her progress. For example, graphical user interface 1300 may include a toggle switch that allow the user to activate a comparison mode within the My Progress screen. In FIG. 14, graphical user interface 1300 has been altered so as to include summary information for two periods of time. Within annotation box 1402, a summary is provided for analyte measurements taken during the most current 90-day period, while in annotation box 1404, a summary is provided for analyte measurements that were taken in the previous 90-day period. As discussed above, the analyte values may include color-coding for measurements that are within, above, or below a target range. In this way, the user may easily keep track of his or her progress over different time periods using the color-coded graphical display.

Returning to FIG. 2, in one or more embodiments of the disclosure, user 102 may grant one or more individuals access to certain CGM-related data, such as analyte measurement data, event data, and sensor data. The identified individual may access this CGM-related data on the individual's own device using an application, such as a “Follower App”, that is configured to provide the CGM-related data of user 102. For example, user 202 may download the Follower App onto device 204. The Follower App may be configured to associate device 204 with user 202. For example, the Follower App may require user 202 to input identification information (e.g., a user ID) and verification information (e.g., a password or biometric data) in order to associate device 204 with user 202. Devices 104 and 106 may similarly be associated with user 102. User 102 may then use device 104 or 106 to grant user 202 access to CGM-related data of user 102.

The grant of access to user 202 by user 102 may be accomplished by device 104 or 106 transmitting access-grant data to remote computer 210. The access-grant data may include user identification information for both user 102 as well as for any individual that user 102 is granting access, and this data may be stored on computer 210. For example, remote computer 210 may be a server that can be accessed by device 204 through the Follower App. Device 204 may transmit user identification and verification information for user 202 to computer 210. The user identification information for user 202 may be compared to user identification information contained in the stored access-grant data. If the identification information for user 202 matches the user identification information provided in the access-grant data, computer 210 may transmit CGM-related data of user 102 to device 204. If the identification information for user 202 does not match any identification information contained in the stored access-grant data, then no CGM-related data of other users will be provided to user 202 via computer 210.

The CGM-related data of user 102 that is transmitted to device 204 may be the subset of the CGM-related data of user 102 that has been selected by user 102 as being accessible to user 202. Accordingly, the Follower App that is running on device 204 will only provide user 202 with the selected subset of the CGM-related data for user 102. For example, user 102 may grant user 202 access to sensor-life data and analyte measurement data for, but not grant user 202 access to event data or may only grant access for certain kinds of events. Any CGM-related data for which user 202 has been granted access can be displayed on device 204 in the same manner as described above. For example, the Follow App may display a sensor-life indication 360 that has an identical appearance to the sensor-life indications 360 discussed above. Like the sensor-life indication 360 displayed on device 102, the sensor-life indication 360 displayed on device 104 may be controlled to switch between a collapsed mode and an expanded mode. In addition, device 104 may display the same sensor-life notifications as those discussed above for device 102. The sensor-life indication 360 may remain visible as an overlay as the user 202 navigates to different screens within the Follower App displayed on device 204.

In one or more embodiments, devices 104 and 204 may receive and display push notifications relating to sensor-life data. For example, computer 210 may determine that the analyte sensor 112 for user 102 is within a predetermined period of time and send a push notification to devices 104 and 204 that provides a statement of how much time remains before analyte sensor 112 expires. For example, computer 210 may transmit push notifications when it is determined that analyte sensor 112 has 3 days, 24 hours, and/or 2 hours remaining before expiration.

As discussed above, the user 102 may replace his or her analyte sensor 112 with a new analyte sensor 112. The new analyte sensor 112 may be paired with a user device, such as device 104. Upon pairing with the new sensor 112, device 104 may transmit sensor-life data of the new analyte sensor 112 to computer 210. Computer 210 may then transmit the sensor-life data of the new analyte sensor 112 to device 204 in accordance with access-grant data that has been previously stored on computer 210. Upon receiving the sensor-life data of the new analyte sensor 112, device 204 may update the displayed sensor-life indication to correspond with the expiration date and time for the new analyte sensor 112.

Returning to FIG. 2, user 202 may use device 204 to track the CGM-related data of a plurality of users 102, each of which are having analyte measurements taken by sensor 112. Each user 102 may grant access user 202 access to at least some subset of CGM-related data in the manner described above. Computer 210 may receive each user's CGM-related data, as provide by each user device 104, and computer 210 may transmit the received CGM-related data of each user to device 204 in accordance with the access that has been granted to user 202 by each user 102. In one or more embodiments, device 204 may include a Follower App that is configured to display the plurality of users 102 for which user 202 has been granted access to their CGM-related data. For example, FIG. 15 is scrollable graphical user interface 1500 that is displayed on device 204 to show all of the users 102 (not shown) that user 202 (not shown) is following. Each user may be listed within an individual user listing 1502. Each user listing 1502 may include identification information, such as the followed user's name, as well as a status indication 1504 that may provide the most recent analyte measurement for the followed user, as well as indicate whether the analyte measurement is increasing, decreasing or stable. Each user listing 1502 may also include a time indication 1506 that indicates the last time data was received for that user. For example, user listing 1502a identifies the followed user as being “John Smith.” The status indication 1504a indicates that John Smith's analyte measurement is at 68 mg/dl and falling, and the time indication 1506a states that this measurement is current, as it was taken “now.” The time indication may identify the number of hours or minutes since the last analyte measurement was received, such as time indication 1506b. If it has been one or more days since the last measurement, the time indication may provide the date and time of the last analyte measurement, as is provided with time indication 1506c.

The status indication 1504 may also indicate if the user's sensor is above or below the sensor limit. For example, status indication 1504d states that the sensor is “HIGH,” meaning that it is above the sensor limit. The user listings 1502 and status indicators 1504 may each be color-coded in the manner described above, so as to indicate if the followed user's most recent analyte measurement is within, above, or below a predetermined range of analyte values. In this way, the user of the Follower App may quickly assess the status of a plurality of users on one display screen. The user listing 1502 may also indicate if no data is available for a user, such as can be seen for user listing 1502e in which the time indication states that no data is available and for which the status indication 1504e does not contain any numeric value. A user listing may 1502 may also contain a status indication 1504 with a high or low glucose alert graphic. For example, user listing 1502f contains a “Low Glucose Alert” along with an exclamation icon.

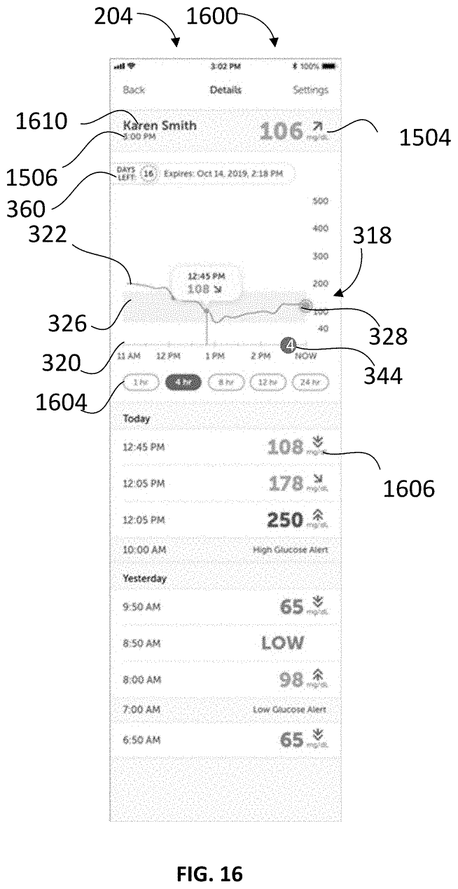

Any user listing 1502 may be selected by the user of the Follower App so as to access detailed information about that user. For example, FIG. 16 is graphical user interface 1600 that may be displayed on device 204 when a particular user listing has been selected. Graphical user interface 1600 may include the features described in connection with graphical user interface 300 of FIG. 3A. For example, graphical user interface 1600 includes sensor life indication 360, historical graph 318, now dot 328, graphical analyte measurements 322, and target analyte band 326, as well as timeline 320. The display also contains the user's name, a time indication 1506 status indication 1504, and a color-coded historical list 1606 of analyte values. This historical list 1606 may provide the analyte values at particular times that have been selected by the user of device 204. In addition, if the user of device 204 has been granted access to event data, historical graph 318 may include event indications, such as event indication 344. The user may zoom in and out of historical graph 318 in the manner described above.

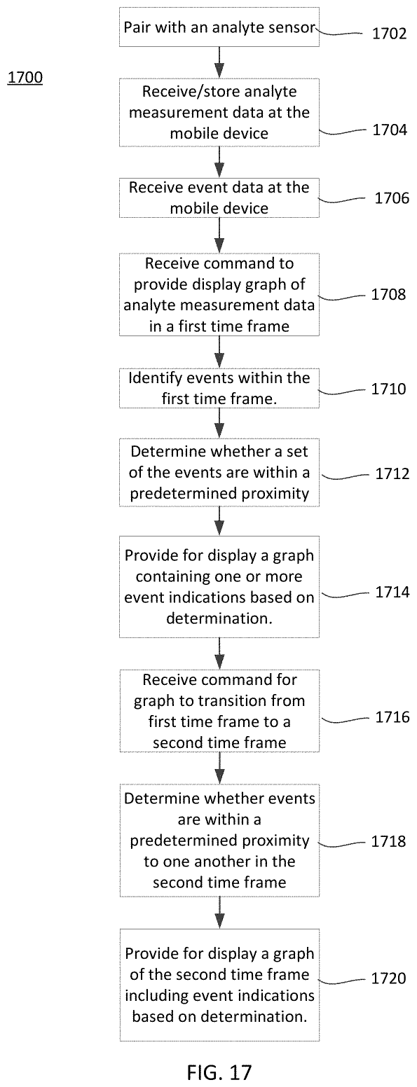

FIG. 17 is a flow diagram 1700 that corresponds to functions that can be taken by one or more processors of the system described in FIGS. 1 and 2 above in order to collect and display analyte data on a mobile device, such as device 104. Multiple processors may be used to perform the disclosed functions, including performing some of the processing on a remote computer, such as computer 210 discussed in FIG. 2.

As described above, the mobile device may pair with an analyte sensor that is worn by a user (Block 1702). The mobile device may receive and store analyte measurement data that has been collected and transmitted by the analyte sensor. (Block 1704). The mobile device may receive event data (Block 1706). As described above, event data may be input by a user of the mobile device, and event data may include CGM-related data, such as information about insulin taken by the user, physical activities, food that has been consumed, and CGM-relevant notes provided by the user. Event data includes data identifying the time of the event, which may include the time the event was recorded. The user of the mobile device may provide a user input command that will cause the mobile device to display analyte measurement data within a historical graph that corresponds to a first time frame. (Block 1708). As described above, the first time frame may be one of a plurality of default time frames selected by the user, or may correspond to a previously viewed time frame by the user. The mobile device may identify a plurality of events that are associated with a time or range of time that corresponds with the first time frame (Block 1710). In Block 1712, a determination is made whether two or more events are within a predetermined proximity to one another within the first time frame. As described above, the predetermined proximity may a proximity of event indications that would correspond to the events within a timeline of the first time frame. The predetermined proximity may also be defined by a range of time between two events. The range of time that constitutes the predetermined proximity can be based on the length of the first time frame. In particular, the range of time used as the predetermined proximity may be correlated to the length of time of the first time frame, so that the longer the first time frame is the longer the range of time will be for predetermined proximity.

Mobile device may provide for display a graph containing one or more event indications based on the determination of the proximity of the identified events. (Block 1714). As described above, an event indication may correspond to more than one event if a determination is made that two or more events are within the predetermined proximity to one another along the timeline of the first time frame. In Block 1716 the mobile device receives a user input consistent with a command for the displayed graph to transition from the first time frame to a second time. In response to the command, the mobile device determines whether events that occurred in the second time frame are within a predetermined proximity to one another with respect to the second time frame. (Block 1718) As discussed above, if the user input corresponds to zooming into the graph, some events that were previously within a predetermined proximity in the first time frame will no longer be within a predetermined proximity in the second time frame. Alternatively, if the user input corresponds to zooming out of the graph, some events will be within a predetermined proximity in the second time frame that were not within a predetermined proximity within the first time frame. In Block 1720, the mobile device provides for display a graph of the second time frame that includes event indications that are based on the determination of the events' proximity to one another. In displaying the event indications in the second time frame, one or more events may be displayed in connection with a new time and analyte measurement within the displayed graph compared to the time and analyte measurement for which the event was displayed in the first time frame.

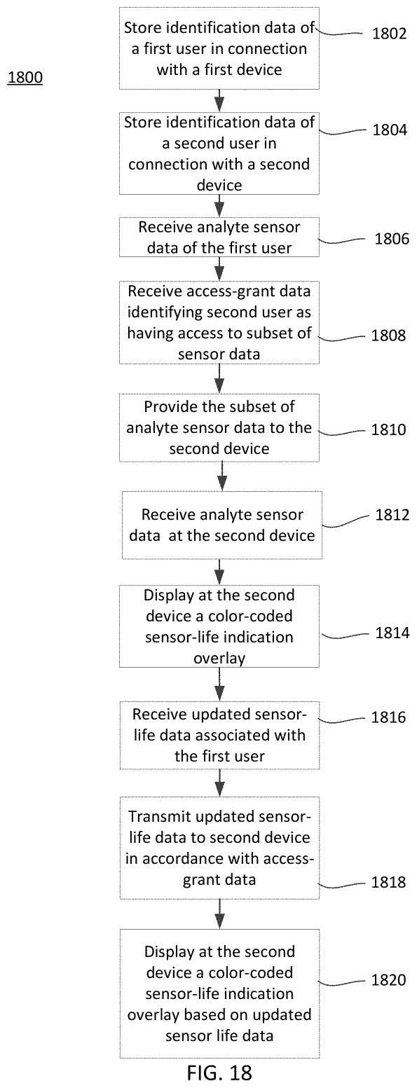

FIG. 18 is a flow diagram 1800 that may be performed by the disclosed systems, such as the system described in FIG. 2. In Blocks 1802 and 1804, one or more processors, such as those of one or more servers, store identification data of a first user in connection with a first device and identification data of a second user in connection with a second device. As described above, this connection of identification data with a device associates the device with a particular user. In Block 1806, one or more servers receive analyte sensor data for an analyte sensor being used by the first user. The analyte sensor data may include analyte measurements, event data, and sensor-life data. In Block 1808, one or more processors receive access-grant data from a device associated with the first user. The access-grant data identifies the second user as having access to a particular subset of the first user's analyte sensor data and event data. The one or more servers transmits the designated subset of the first user's analyte sensor data to the second device in accordance with the access-grant data (Block 1810). The transmission of the subset of the analyte sensor data may be pushed automatically to the second device or may be provided in response to a request from the second device.

In Block 1812, the second device receives the transmitted analyte sensor data of the first user, and in Block 1814, the second device provides for display a sensor-life indication overlay in accordance with the analyte sensor data. As described above, the sensor-life indication may be color-coded so as to designate the units of time that the sensor-life indicator represents. The one or more servers may receive updated sensor-life data from a device associated with the first user (Block 1816). For example, the first user may pair the first device with a new analyte sensor. Upon pairing with a new sensor, the first device may transmit analyte sensor data for the new analyte sensor to the one or more servers. In Block 1818, the one or more servers transmit the updated sensor-life data to the second device in accordance with stored access-grant data. The second device may then display an updated sensor-life indication based on the updated sensor-life data (1820).

Embodiments are described herein with reference to flowchart illustrations and/or block diagrams of methods, apparatus (systems), and computer program products according to embodiments. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

1. A system for displaying analyte measurements comprising:
a display;
one or more memories for storing analyte measurement data and event data; and
one or more processors configured to:
access analyte measurement data and event data from the one or more memories;
determine a first time frame for which to display analyte measurement data as part of a graph;
identify a plurality of events within the event data that occurred within the first time frame;
determine, based on times associated with the plurality of events, whether a set of events are within a predetermined proximity in the graph having the first time frame; and
provide for display the graph containing one or more event indications corresponding to the plurality of events, wherein a set of events are combined to be represented by a first event indication if it is determined that the set of events are within the predetermined proximity, and wherein each of the one or more event indications are associated with an analyte measurement within the graph.

2. The system of claim 1, wherein the one or more processors are further configured to:
identify a first user input as corresponding to a command for the graph to transition from the first time frame to a second time frame, wherein the second time frame is a shorter period of time than the first time frame;
determine that the set of events within the graph in the second time frame are outside of the predetermined proximity; and
in response to the first user input, automatically provide for display the graph in the second time frame, wherein the set of events are displayed in connection with the graph as at least two separate event indications.

3. The system of claim 1, wherein the one or more processors are further configured to:
identify a second user input as corresponding to a command for the graph to transition from the first time frame to a third time frame, wherein the third time frame is a longer period of time than the first time frame;
determine that one or more additional events within the graph in the third time frame are within of the predetermined proximity of the set of events; and
in response to the second user input, automatically provide for display the graph in the third time frame, wherein the set of events and the additional events are displayed in connection with the graph as a single combined event indication.

4. The system of claim 3, wherein the single combined event indication is associated with a single analyte measurement.

5. The system of claim 1, wherein the events within the set of events are associated with different times and wherein the first event indication is placed within the graph at a position corresponding to a median time period between the different time periods.

6. The system of claim 1, wherein the first event indication includes a symbol indicating the number of events with which it is associated.

7. The system of claim 1, wherein the one or more processors are further configured to
identify user input selecting a particular event indication displayed with the graph; and
in response to the selection the particular event indication, providing for display additional information relating to the one or more events associated with the event indication, wherein the additional information includes a trend graph of analyte measurements for each of the one or more events.

8. The system of claim 7, wherein each trend graph displays a range of analyte measurements corresponding to predetermined periods of time before and after each of the one or more events.

9. The system of claim 1, wherein the one or more processors are further configured to:
identify a sensor that is configured to provide the analyte measurements;
determine a period of time for which the sensor will remain operational; and
provide for display a sensor-life indication in the same display as the graph, wherein the sensor-life indication includes a number indicating the period of time for which a sensor will remain operational, and wherein the sensor life indication is color-coded to indicate the units of time represented by the number.

10. The system of claim 9, wherein the number is surrounded by a plurality of ticker marks corresponding to the units of the time period represented by the number.

11. The system of claim 1, wherein the one or more processors are further configured to:
compare a most-recent analyte measurement to one or more threshold values; and
provide for display, within the graph, a current measurement indication having a color, wherein the color is based on the comparison of the analyte measurement with the one or more threshold values.

12. The system of claim 11, wherein the one or more processors are further configured to:
determine if the one or more processors are in communication with an operational sensor, and wherein the current measurement indication pulsates if the one or more processors are in communication with an operational sensor.

13. The system of claim 12, wherein the one or more processors are further configured to:
identify a user selection of a particular point within the graph provided for display;
determine an analyte measurement associated with the particular point; and
provide for display a color-coded analyte measurement value, wherein the color corresponds to the relation of the analyte measurement value to one or more thresholds.

14. A method for displaying analyte measurements comprising:
Storing, by one or more processors, analyte measurement data and event data;
accessing, by the one or more processors, analyte measurement data and event data from the one or more memories;
determining, by the one or more processors, a first time frame for which to display analyte measurement data as part of a graph;
identifying, by the one or more processors, a plurality of events within the event data that occurred within the first time frame; and
determining, by the one or more processors, whether, based on times associated with the plurality of events, a set of events are within a predetermined proximity in the graph having the first time frame;
providing for display, by the one or more processors, the graph containing one or more event indications corresponding to the plurality of events, wherein a set of events are combined to be represented by a first event indication if it is determined that the set of events are within the predetermined proximity, and wherein each of the one or more event indications are associated with an analyte measurement within the graph.

15. The method of claim 14, further comprising:
identifying, by the one or more processors, a first user input as corresponding to a command for the graph to transition from the first time frame to a second time frame, wherein the second time frame is a shorter period of time than the first time frame;
determining, by the one or more processors, that the set of events within the graph in the second time frame are outside of the predetermined proximity; and
in response to the first user input, automatically provide for display, by the one or more processors, the graph in the second time frame, wherein the set of events are displayed in connection with the graph as at least two separate event indications.

16. The method of claim 14, further comprising:
identifying, by the one or more processors, a second user input as corresponding to a command for the graph to transition from the first time frame to a third time frame, wherein the third time frame is a longer period of time than the first time frame;
determining, by the one or more processors, that one or more additional events within the graph in the third time frame are within of the predetermined proximity of the set of events; and
in response to the second user input, automatically provide for display, by the one or more processors, the graph in the third time frame, wherein the set of events and the additional events are displayed in connection with the graph as a single combined event indication.

17. The method of claim 16, wherein the single combined event indication is associated with a single analyte measurement.

18. The method of claim 14, wherein the events within the set of events are associated with different times and wherein the first event indication is placed within the graph at a position corresponding to a median time period between the different time periods.

19. The method of claim 14, wherein the first event indication includes a symbol indicating the number of events with which it is associated.

20. The method of claim 14 further comprising:
identifying, by the one or more processors, user input selecting a particular event indication displayed with the graph; and
in response to the selection the particular event indication, providing for display, by the one or more processors, additional information relating to the one or more events associated with the event indication, wherein the additional information includes a trend graph of analyte measurements for each of the one or more events.

21. The method of claim 20, wherein each trend graph displays a range of analyte measurements corresponding to predetermined periods of time before and after each of the one or more events.

22. The method of claim 14, further comprising:
identifying, by the one or more processors, a sensor that is configured to provide the analyte measurements;
determining, by the one or more processors, a period of time for which the sensor will remain operational; and
providing, by the one or more processors, for display a sensor-life indication in the same display as the graph, wherein the sensor-life indication includes a sensor-life number indicating the period of time for which a sensor will remain operational, and wherein the sensor life indication includes a color code to indicate the units of time represented by the sensor-life number.

23. The method of claim 22, wherein the sensor-life number is surrounded by a plurality of ticker marks corresponding to the units of the time period of the number.

24. The method of claim 14, further comprising:
wherein the graph provided for display includes an enlarged point associated with the most recent analyte measurement, and wherein the enlarged point

25. The method of claim 14, further comprising:
comparing, by the one or more processors, a most-recent analyte measurement to one or more threshold values; and
providing for display, by the one or more processors, within the graph, a current measurement indication having a color, wherein the color is based on the comparison of the analyte measurement with the one or more threshold values.

26. The method of claim 25, further comprising:
Determining if the one or more processors are in communication with an operational sensor, and wherein the current measurement indication includes an animation if the one or more processors are in communication with an operational sensor.

27. The method of claim 26, further comprising:
identifying, by the one or more processors, a user selection of a particular point within the graph provided for display;
determining, by the one or more processors, an analyte measurement associated with the particular point; and
providing for display, by the one or more processors, a color-coded analyte measurement value, wherein the color corresponds to the relation of the analyte measurement value to one or more thresholds.
